(19) United States
(12) Patent Application Publication
(10) Pub. No.: US 2026/0355376 A1
(43) Pub. Date: Nov. 12, 2026

(54) NEEDLEFREE VASCULAR ACCESS CONNECTORS

(71) Applicant: CleanSite Medical, Inc., Solana Beach, CA (US)

(72) Inventors: John GRANT, Solana Beach, CA (US); Daniel M. CHAMBERS, San Diego, CA (US); Rick PLUTH, Solana Beach, CA (US)

(73) Assignee: CleanSite Medical, Inc., Solana Beach, CA (US)

(21) Appl. No.: 19/670,922

(22) Filed: May 7, 2026

Related U.S. Application Data
(60) Provisional application No. 63/801,796, filed on May 7, 2025.

(51) Int. Cl. A61L 2/26; A61L 2/18; A61L 101/34; A61L 103/15

(52) U.S. Cl. CPC A61L 2/26; A61L 2/18; A61L 2101/34; A61L 2103/15

(57) ABSTRACT

Improved cleansing and capping devices for cleansing and, if desired, capping, needlefree connectors, particularly luer access devices such as needlefree vascular access connectors, and methods for using said devices, are described. The devices of the invention each include interconnected inner and outer housings that a user can rotate independently, even after the device has been securely yet removably attached to, for example, the threaded valve region of a needlefree connector. Such rotation allows a compressible cleansing matrix secured in the device (preferably in a well in the outer housing and containing a liquid disinfectant such as 70% IPA solution) to be used to actively cleanse potentially contaminated or soiled surface(s) of the valve region of a needlefree connector.

### REFERENCE TO RELATED APPLICATIONS

This application claims the benefit of and priority to commonly owned U.S. provisional patent application serial no. 63/801,796 (attorney docket no. CSM-0025-PV2), filed 7 May 2025, which priority application is hereby incorporated by reference in its entirety for any and all purposes.

### INCORPORATION BY REFERENCE

This application hereby incorporates by reference for any and all purposes the entirety of commonly owned U.S. patent no. 11,065,431.

### TECHNICAL FIELD OF THE INVENTION

This invention is directed to improved cleansing devices for cleaning, disinfecting, and capping medical devices, particularly luer access devices such as needlefree, valved connectors (NFCs), and methods for making and using such articles.

### BACKGROUND OF THE INVENTION

### Introduction

The following description includes information that may be useful in understanding the present invention. It is not an admission that any such information is prior art, or relevant, to the presently claimed inventions, or that any publication specifically or implicitly referenced is prior art.

### Background

In the medical field, and in particular the area of infusion of fluids or aspiration of fluids to or from a patient, there remains a need to prevent the transmission of pathogens into or onto a patient from a potentially contaminated surface of a medical device such as a luer access device, for example, a needlefree, valved connector (NFC). Pathogens include microorganisms such as bacteria, fungi, and viruses, the transmission of which into a patient may result in an infection that could be life-threatening. Specific to healthcare settings, the term “nosocomial infection” describes those infections that originate from or occur in a hospital or hospital-like setting. In the U.S., nosocomial infections are estimated to occur in at least 5% of all acute care hospitalizations. The estimated incidence is more than two million cases per year, resulting in significant morbidity, mortality, and an expense. Indeed, nosocomial infections are estimated to more than double the mortality and morbidity risks of any admitted patient, and likely result in about 100,000 deaths a year in the United States alone. Common sites for the transmission of contaminating microorganisms into a patient’s bloodstream are found on medical devices such as luer access devices, vials, needlefree (or needle free) valves, and the injection ports of vessels, tubing, and catheters. The incidence of such infections in patients is increasing, and infection control practitioners (ICPs) often cite improper cleansing of sites as a major source of such infections.

As described above, patient exposure to pathogens and infectious reagents (e.g., pathogenic bacteria, viruses, fungi, etc.) in medical settings (e.g., hospitals, out-patient surgery centers, home-care settings, etc.) is a matter of serious concern. One route of exposure to such reagents is the opening made in skin provided by the bore of needle, cannula, or other similar device used to provide access to a patient’s vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC). All told, in the U.S. approximately (28),000 patients die annually from hospital-acquired infections that result from PIVC and CVC use, and many times that number are made seriously ill but survive. Costs associated with the care and treatment of patients that develop infections due to PIVC and CVC use is estimated to exceed $4 billion annually in the U.S. alone.

In hospital settings today, occupational health and safety regulations designed reduce the risk to health care workers from needle stick and similar injuries have resulted in the deployment of needlefree medical valves (also referred to herein as “needlefree connectors” or “NFCs”) whenever possible. Currently, more than 1 billion NFCs are used annually in hospitals throughout the U.S. Needlefree connectors are used primarily in conjunction with PIVC and CVC devices and associated IV administration and extension sets, which may contain from as few as one to as many as 3, 4, 5, or more NFCs.

The widespread use of needlefree connectors in acute medicine has contributed to a marked increase in the incidence of healthcare-associated infections (HAIs), particularly blood stream infections (BSIs). To reduce the risk of infection from a needlefree connector contaminated with microorganisms, standard practice today requires that a nurse or other healthcare worker clean (or “scrub”) the surface of NFC by vigorously rubbing those of its exterior surfaces in the fluid path with a sterile alcohol swab or wipe immediately prior to making a fluid connection to the NFC, for example, by attaching a syringe to the NFC’s threaded valve portion to deliver a medication via a PIVC already connected to the patient. Given the magnitude of the mortality and morbidity associated with HAIs, particularly with regard to central line-associated blood stream infections (CLABSIs), and the large number of blood stream infections that result from PIVC and CVC use (so-called “peripheral line- associated blood stream infections” (PLABSIs) and central line-associated blood stream infections (CLABSIs), respectively), a long-recognized yet significant unmet need exists for articles or devices that can be used to reduce or eliminate the risk of initiating an HAI merely by accessing a patient’s vasculature through a needlefree valve component of a PIVC or CVC inserted into a blood vessel of a patient.

Traditionally, and as noted above, cleaning, cleansing, or disinfecting a potentially contaminated NFC surface involved a protocol of alcohol swabbing prior to making the necessary connections to the site. Alcohol swabs are typically small pads of cotton gauze soaked in isopropyl alcohol (IPA), packed individually in foil packages to prevent evaporation of the IPA from the swab prior to use. Properly used, the package is opened at or near the site to be swabbed. With gloved hands, the swab is removed by a nurse or other healthcare provider and used to scrub the top and side surfaces of the valve portion of the NFC to be connected. After use, the swab and foil package are discarded and the cleansed valve portion of the NFC is allowed to dry, usually 20-30 seconds, immediately prior to making any connection. This “drying” period is important because, as the IPA dries, it breaks open the cellular walls of microorganisms, thereby killing them.

Unfortunately, because of increased duties and responsibilities, shrinking nursing staffs, and inadequate training, alcohol swabbing (or scrubbing) is often not performed or is poorly executed. A poorly swabbed site can carry microorganisms that, if allowed to enter a patient’s body, can cause serious, and potentially life-threatening, infection. In addition, supervisory oversight is nearly impossible, because unless a supervisor actually observes swabbing as it is performed, the supervisor cannot know whether or not the scrubbing procedure was done properly or performed at all. Indeed, reported compliance with such “scrub the hub” protocols has been as low as 10%. Further, without at least a sufficient microscopic examination for microbial residue (e.g., biofilm), there may be no evidence of “scrubbing the hub” being performed.

Thus, a significant need still exists for devices and techniques cleanse (i.e., clean and disinfect) sites on medical devices prior to their use with or connection to patients, and which eliminate technique-related and training issues and provide an unequivocal indicator that a site is clean prior to accessing a patient's vascular system.

### Definations

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” refers to approximately a +/-10% variation from the stated value. It is to be understood that such a variation is always included in any given value provided herein, whether or not it is specifically referred to.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “species”, when used in the context of describing a particular compound or molecule species, refers to a population of chemically indistinct molecules.

### SUMMARY OF THE INVENTION

The object of the invention is to address these long-standing but still unmet needs. This invention addresses these needs by providing patentable, single-use cleansing (cleaning and disinfecting) and capping devices or articles that can be used to effectively and efficiently cleanse/disinfect and cap, and preferably sterilize, exposed surfaces of medical articles such as luer access devices, particularly needlefree connectors, particularly the accessible surface(s) of the threaded valve portions of needlefree connectors, particularly those surfaces (valve surfaces, threads, etc.) that may become contaminated with pathogens or other infectious reagents and which form part of the fluid communication pathway between an external fluid source (e.g., a medicine filled syringe with a male luer fitting, an IV bag, etc.) and a patient’s blood stream. In the context of the invention, “cleanse” encompasses cleaning, disinfecting, sanitizing, and/or sterilizing, whereas “capping” refers to using a device, i.e., a “cap”, to cover a surface, or set of surfaces, of an NFC so as to limit or prevent exposure of such surface(s) to the environment (e.g., the air circulating in a hospital’s intensive care unit, the microbiome resident on a patient’s skin, clothing, bedding, unclean fingers, etc.) for a period longer than necessary to cleanse the desired surface(s) of a needlefree connector.

Thus, in one aspect, the invention provides cleaning, disinfecting, and capping devices for medical devices such as luer access devices, including needlefree, valved vascular access connectors (NFCs)., especially the threaded valve portions of such connectors.

Such devices typically comprise an inner housing disposed in an outer housing, a compressible cleansing matrix with a liquid disinfectant disposed therein, and optionally, a removable seal. Such devices are intended for single use, are preferably sterilized, and can be configured as individually sealed units or arrayed as a plurality of units sealed using a single piece of seal or lid material.

More specifically, the inner housing is configured for secure, removable attachment to a threaded valve portion of a needlefree connector, which thus allows the entire device to be removable yet securely attached to a needlefree connector. The inner housing includes a central bore that includes oppositely disposed first (e.g., top) and second (e.g., bottom) openings. The first and second openings can be the same size (e.g., by diameter) or, preferably, be of different sizes. In certain preferred embodiments, the first opening is larger than the bottom opening. The central bore may have the same or different diameter over its length. In some embodiments, the upper portion of the central bore has a larger diameter than the lower portion of the central bore, in which event there may be a step or similar structure in the interior surface of the central bore at the transition between the upper and lower regions of the central bore.

The inner housing is configured to allow the device to be screwed onto and unscrewed from the threaded valve portion of the connector while at the same allowing a compressible cleansing matrix attached to the outer housing to protrude into the central bore through the first opening so as to allow contact between the compressible cleansing matrix and a valve surface of the threaded valve portion of the needlefree vascular access connector when the device is screwed onto the threaded valve portion of the connector. This is preferably accomplished by including threads (or thread tabs) on the interior surface of at least the lower part of the central bore.

The inner housing further includes a downwardly projecting, flexible, circumferential biasing element protruding from its outer surface, an outer housing retaining element, and an outer housing engaging element disposed on an upper surface of the inner housing. The biasing element is designed to be compressed from a first or “free” position to a compressed or second position, such movement being inward, toward the outer wall of the central bore, as happens during assembly of the inner and outer housings into a rotatable subassembly. Such inward movement causes the compression gap between the biasing element and adjacent portion of the central bore to become reduced. Upon completing assembly of the rotatable subassembly, the biasing element of the inner housing rebounds into a partially compressed third position intermediate between the first and second, or free and compressed, positions. As will be appreciated, the compression gap between the biasing element and central bore wall is maximal when the biasing element is in its first or “free” position, is smallest when the biasing element is in its second position (as happens during assembly of the inner and outer housings), and is of intermediate size in a completed inner and outer subassembly or in the finished device (with the inner housing rotatably assembled into the outer housing, the compressible matrix being operably connected to the outer housing and protruding into the central bore of the inner housing, and with liquid disinfectant being present in addition to a peelable lid sealing the device). In the third position, the biasing element is advantageously under tension when it contacts the opposing surface of the other housing’s retaining element.

The inner housing’s outer housing retaining element is disposed in an outer surface of the biasing element and is configured to engage a complementary inner housing retaining element that extends from an inner surface of the outer housing’s sidewall. The inner housing’s outer housing retaining element is also sized to allow the outer housing to vertically move in relation to the inner housing, for example, as occurs when the outer housing is pushed upward by the compressible cleaning matrix into a neutral position useful for scrubbing an NFC after a user attaches the device to a valve region on an NFC. The inner housing also includes an outer housing engaging element, which is preferably disposed on an upper surface of the inner housing.

In some embodiments, the inner housing includes at least one fluid path disposed on an inner surface of a sidewall.

In some embodiments, the inner housing has a height that is less than the depth of the cavity of the outer housing

A device according to the invention also includes an outer housing rotatably connected to the inner housing. The outer housing comprises a cavity in which the inner housing is rotatably disposed. The outer housing is configured to (A) retain, via complementary retention elements on facing surfaces of the inner and outer housings , the inner housing in the cavity, and (B) engage and disengage from the inner housing so as to allow the outer housing to (i) retain the inner housing and be rotated independently of the inner housing in both a clockwise direction and a counter-clockwise direction by a user when complementary engaging elements on the inner and outer housings are not engaged and (ii) retain the inner housing and be rotated in unison with the inner housing by the user when the outer housing and the inner housing are engaged.

In preferred embodiments, the outer housing further comprises a matrix well that extends into the cavity from the inner surface of the top of the outer housing to securely retain the compressible cleansing matrix, wherein an inner surface of the matrix well optionally includes one or more matrix retention splines disposed thereon

A device according to the invention also includes a compressible cleansing matrix attached to the outer housing and protruding into the inner housing, wherein the compressible cleansing matrix rotates with the outer housing. Advantageously, a liquid disinfectant is disposed in the compressible cleansing matrix, optionally isopropyl alcohol, advantageously a solution of about 70% isopropyl alcohol and water. In some embodiments, the liquid disinfectant further comprises chlorhexidine, for example, chlorhexidine gluconate.

In preferred embodiments, a device according to invention, the compressible cleansing matrix is configured to contact at least a valve surface of the threaded valve portion of the needlefree connector when the device is threaded onto the threaded valve portion of the needlefree connector. In some of these embodiments, the compressible cleansing matrix is configured to also contact an additional surface of the threaded valve portion of the needlefree connector when the cleansing and capping device is threaded onto the threaded valve portion of the needlefree connector

In some embodiments, a device according to invention further comprises a seal between the inner housing and the outer housing.

In preferred embodiments, a device according to invention further comprises a removable lid or seal to seal device from an external environment.

In preferred embodiments, a device according to invention is sterilized.

A related aspect of the invention concerns a cleansing and capping device according to invention threaded on to a threaded valve portion of a needlefree connector. Another aspect of the invention concerns methods of cleaning and/or disinfecting needlefree connector. Such methods generally include securing a device according to the invention to a threaded valve portion of the needlefree connector and then rotating the outer housing in relation to the first housing cleanse (e.g., clean and/or disinfect) one or more surfaces of the threaded valve portion of the needlefree connector. Such methods preferably involve removing the cleansing and capping device of the invention from the threaded valve portion of the needlefree connector within not less than about 30 seconds of attaching the cleansing and capping device to the needlefree connector. Dwell times of about 30 seconds to about 7 days are envisioned.

In preferred embodiments of such methods, cleansing substantially disrupts any microbial contamination, for example, microbial biofilm or other microbial contamination that may exist on surfaces contacted by the compressible cleansing matrix. If desired, the cleansing and capping device can be left in place (typically after cleansing the needlefree connector attached thereto) in order to cap the needlefree connector until it is further accessed, thereby minimizing exposure of capped exterior surfaces of the NFC to potential pathogen contamination (and biofilm formation) from the surrounding environment. Lids or seals are typically installed during manufacture of a cleansing and capping device of the invention. In those embodiments where the cleansing and capping devices are sterilized during manufacture (e.g., by irradiation, exposure to ethylene oxide, etc.), lids or seals are preferably applied prior to packaging and sterilization.

Features and advantages of the invention will be apparent from the following detailed description, and appended claims.

### BRIEF DESCRIPTION OF THE DRAWINGS

These and other aspects will now be described in detail with reference to the following drawings. Unless otherwise indicated, it is understood that the drawings are not to scale, as they are intended merely to facilitate understanding of the invention as opposed to specific dimensions, etc. In the drawings, like numbers in two or more drawings represent like elements.

FIG. 1 shows exploded and assembled views of a representative embodiment of a cleansing and capping device (1) according to the invention, including an outer housing (4), an inner housing (7), and a compressible cleansing matrix (10). In this embodiment, the inner housing retaining element of the outer housing is a circumferential ridge that rises from the inner (or interior) surface of the outer housing’s sidewall that engages with the outer housing retaining element of the inner housing. Here, the outer housing retaining element is configured as a circumferential channel disposed on the outer (or exterior) surface of the spring/biasing element of the inner housing. The height of the channel is several times that of the outer housing’s ridge-shaped inner housing retaining element. This configuration allows the outer housing to vertically translate in relation to the inner housing, which allows the engaging elements of the inner and outer housings to move between engaging and disengaging positions. When these engaging elements are engaged, the inner and outer housings rotate together (or in unison); when disengaged, the outer housing can rotate without the inner housing rotating.

FIG. 2 shows exploded and assembled views of another representative embodiment of a cleansing and capping device of according to the invention. In this embodiment, the inner housing retaining element of the outer housing is configured as a circumferential channel disposed in the inner (or interior) surface of the outer housing’s sidewall while the outer housing retaining element of the inner housing is configured as a circumferential ridge that rises from the outer (or exterior) surface of the spring/biasing element of the inner housing,

FIG. 3 shows exploded views of a representative embodiment of a cleansing and capping device as well as an assembled view of the device attached to a needlefree connector.

The details of one or more embodiments are set forth in the accompanying drawings and the description below. Other features and advantages will be apparent from the description and drawings, and from the claims.

### DETAILED DESCRIPTION

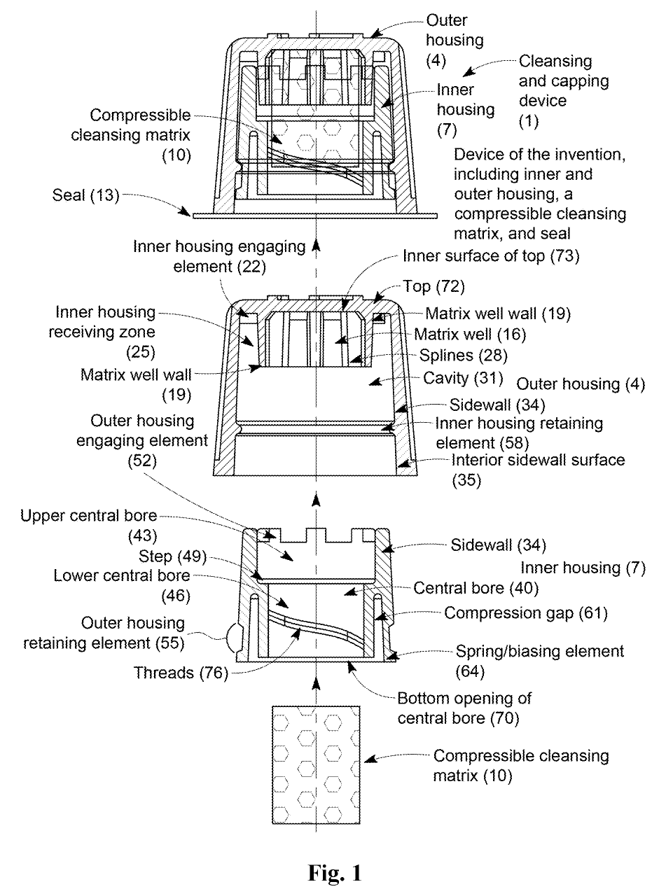

In the following detailed description, reference is made to the accompanying FIGS. 1-3, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here.

This invention concerns patentable, preferably sterile, single-use cleansing (cleaning and disinfecting) and capping devices that can be used to effectively and efficiently clean, disinfect, and preferably sterilize, exposed surfaces of needlefree connectors, particularly those of luer access devices such as needlefree medical valves that at times become part of a fluid communication pathway for introduction of fluids (e.g., IV fluids, blood, plasma, medicines, etc.) into a patient, as these surfaces are at risk for contamination with pathogens and infectious reagents such as bacteria, fungi, and viruses. “Single-use” (or “single purpose”) refers to an article or device suitable for one use or purpose only, as distinguished from “dual” or “multiple” use or purpose devices. Thus, in the context of the invention, a “single-use” cleansing and capping device is one that is useful for cleansing, for example, a needlefree medical valve. After the cleansing operation, the device may, if desired, be left in place on the needlefree connector until a subsequent fluid connection is made in order to prevent recontamination of the connector’s cleansed surface(s), as would occur if the cleansing and capping device of the invention was removed immediately following “scrubbing” of the connector without a fluid connection then being made. After removal, the device of the invention is preferably discarded. Prior to removal, however, the cleansing and capping device can again be used to cleanse the capped surface(s) of the needlefree connector.

In general, a cleansing and capping devices of the invention include a first, often an inner, housing configured to allow the device to be screwed onto and unscrewed from the threaded valve portion of an NFC; a second, often an outer, housing that retains the first (e.g., inner) housing but which a user can, when desired, rotate independently of the first (e.g., inner) housing to provide scrubbing or disinfecting action; and a compressible cleansing matrix preferably impregnated with a disinfectant, for example, a 70% IPA solution. The device also preferably includes an easily removable seal to maintain sterility and prevent loss of the disinfectant after the device is assembled until such time as it is used in the field to cleanse and/or cap an NFC. In some embodiments, several cleansing and capping devices according to the invention, e.g., 2,3, 4, 5, 6, 7, 8, 9, 10, or more can be sealed onto a single piece of sealing or lidding material to provide a strip of devices of the invention that can, for example, be hung from an IV pole at a patient’s bedside.

As described, the devices of the invention include an inner housing. In some preferred embodiments, the inner housing is comprised of a sidewall that bounds a central, interior (preferably cylindrical) bore that spans between oppositely disposed first and second (or upper and lower, respectively) openings. In many of these embodiments, the first (upper) opening is sized to allow the compressible cleansing matrix resident at least in part in a matrix well or otherwise attached to the inner surface of the outer housing to protrude into and through the opening into the inner housing’s central bore so that the compressible cleansing matrix can engage one or more exterior surfaces of a needlefree connector when the cleansing and capping device is secured to the connector. The second (lower) opening of the inner housing is sized to allow the threaded valve portion of a needless connector to be cleansed and/or capped to be inserted into the device of the invention. The interior wall of the inner housing’s central bore includes one or more thread-engaging tabs or threads (often helical), preferably two (or more) oppositely disposed (or otherwise spaced) thread-engaging tabs or threads, preferably beginning at or near the interior surface of the central bore of the inner housing’s lower opening. The thread-engaging tab(s) or thread(s) is(are) configured to engage a complementary threaded region on the exterior surface of, for example, a needlefree connector such that the cleansing and capping device can, via association of the thread tabs or threads on the interior of the inner housing’s central bore with complementary threads on the threaded portion of a needlefree connector, be securely threaded onto (or otherwise removably connected with) the targeted threaded portion of a needlefree connector for cleansing and, if desired, capping. Luer threads are preferred, as many medical fittings, including many needlefree connectors, are designed with luer thread fittings.

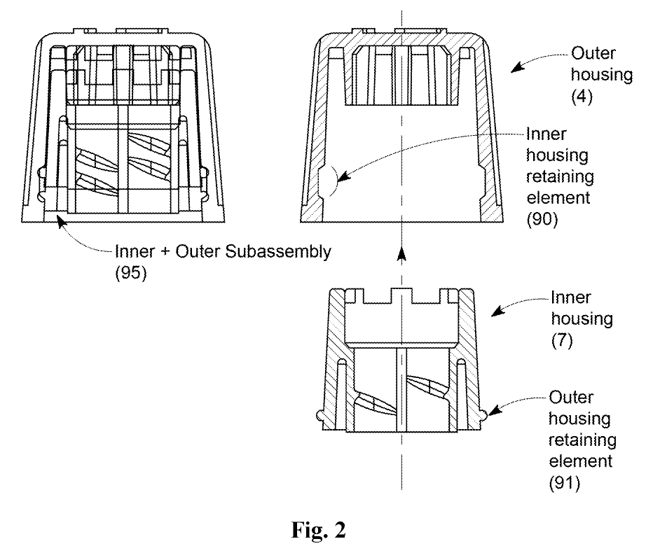

In some preferred embodiments, the outer surface of the inner housing includes an outer housing-retaining region or element (e.g., a channel or recess) that allows the inner housing to be retained in the outer housing via association with one or more complementary structures (e.g., a circumferential ridge or flange (or spaced ridge or flange elements) or skirt or other suitable engaging elements) on the inner (or interior) surface of a portion of the sidewall of the outer housing that interfaces with the opposing surface of the inner housing when the inner and outer housings are assembled together into an inner and outer housing assembly or subassembly. In some embodiments, structures such as circumferential flanges or skirts can also act as a seal, advantageously a fluid seal, between the inner and outer housings. In other preferred embodiments, the outer and inner retaining elements of the inner and outer housings may be reversed (see, e.g., the embodiments depicted in FIGS. 1 and 2).

Preferably, such configurations of complementary retaining elements allow for smooth, low friction movement, for example, rotation and/or vertical translation, of the outer and inner housings in relation to each other during certain operations, for example, during a disinfection procedure of a needlefree connector (when the outer housing is rotated in relation to the inner housing), when the device is being threaded onto or disengaged from the threaded valve portion of a needlefree connector (when the outer housing moves vertically in relation to the inner housing (down in the context of device attachment to an NFC; up in the context of detachment from an NFC)), etc.

Other portions (or areas) of the outer surface of the inner housing further includes one or more outer housing engaging elements or regions designed to associate with one or more inner housing engaging elements or regions disposed on the interior or inner surface of the outer housing. Examples of such elements include, for example, a circumferential band of spaced teeth or teeth-like elements protruding from the exterior upper surface of the inner housing that defines the first (upper) opening of the central bore of the inner housing, which teeth (or other suitable engaging structures) can be engaged by complementary structures arrayed on an adjacent interior surface of the outer housing when the housings are assembled into a functional subassembly.

In some preferred embodiments, the upper exterior surface of the inner housing includes an outer housing-engaging region that includes one or more structures that allow the inner housing to mechanically engage complementary structures (e.g., pawls or other suitable engaging elements) on the interfacing portion of the inner (or interior) surface of the top of the outer housing in the region, for example, between the side wall and matrix well so that when the complementary engaging elements of the inner and outer housings are brought together in an engaging fashion, the engaging elements of the inner and outer housings engage, allowing the inner and outer housings to rotate in unison, as would be required, for example, during attachment and detachment operations between a device of the invention and an NFC. Certain preferred embodiments of outer housing-engaging structures include spaced teeth (or other suitable engaging elements) arrayed on the top or upper surface of the inner housing’s preferably cylindrical sidewall. As will be appreciated, when a device of the invention is securely yet removably attached to an NFC without an external attachment or removal force being applied (for example, so as to move the outer housing down or up, respectively, in relation to the inner housing), such inner and outer housing engaging elements are typically unmated or disengaged, a user can rotate or spin the outer housing in relation to the inner housing, as is, for example, done during a cleansing or disinfecting operation of the NFC to which the device of the invention is attached. Thus, when the cleansing and capping device is secured to a needlefree connector, preferably by a user threading the device on to an NFC until the device is tightly attached (advantageously tactily signaled, for example, by a “hard stop” sensation experienced by the user) when such engaging elements are not functionally associated (or mated or otherwise engaged), a user can rotate the outer housing (and compressible cleansing matrix operably connected thereto) in relation to the inner housing and connected needlefree connector. On the other hand, in such embodiments, when the complementary elements on the inner surface of the top of the outer housing and the upper surface of the top of the inner housing are engaged (in whole or even partially), such as when a user applies downward or upward twisting pressure to the device to place it on or remove it, respectively, from a needlefree connector, the inner and outer housings rotate together, allowing, for example, the cleansing and capping device to be attached to or removed from the NFC.

In other embodiments, the inner and outer housing engaging elements of the outer and inner housings can be disposed in different locations in the outer and inner housings. Preferably, the interfacing engaging elements of the inner and outer housings are configured such that when an axial engaging force applied to the device of the invention during attachment or detachment (or removal) from an NFC the engaging elements of the housings engage, thereby allowing the engaged housings to rotate together as a user attaches or detaches the device from, for example, an NFC.

Advantageously, one or both of the inner and/or outer housings can also include an NFC sealing member configured to provide a fluid tight seal between the cleansing and capping device of the invention and a needlefree connector connected thereto. In some embodiments the NFC sealing member is an O-ring (or comparable seal) preferably disposed in a channel, recess, or the like formed in the inner surface of the wall of the inner housing proximate to the second (lower) opening, typically below the thread-engaging tab(s)(or threads). In some embodiments, the inner housing engaging element on the interior surface of the outer housing can act as a seal when it engages the complementary retaining element (e.g., a channel, recess, etc.) on the exterior surface of the inner housing.

As will be understood, the devices of the invention include an outer housing adapted or configured to retain the inner housing therein such that, when the device is attached to a needlefree connector, under certain conditions the outer housing can rotate (preferably about its central axis) in relation to the inner housing. Any suitable configuration of complementary mechanical or structural features or elements on facing or opposing (or interfacing) surfaces can be used to provide retention of the inner housing inside the outer housing’s main cavity and to allow for engagement and disengagement of the outer housing from the inner housing in order to allow the outer housing to be rotated in relation to the inner housing when the device is attached to a needlefree connector and a user desires to cleanse the corresponding surface(s) of the NFC using a device according to the invention.

In some embodiments, when a device of the invention is attached to a needlefree connector (NFC), the inner and outer housings adopt a disengaged, neutral, or rotating configuration relative to each other such that a user can rotate the outer housing in relation to the inner housing to perform a cleansing operation on the valve portion of the NFC to which the device is attached. Such a disengaged, neutral, or rotating configuration can be achieved by any suitable approach, including by providing complementary engaging elements or structures on adjacent surfaces of the inner and outer housings that under certain conditions, for example, when the outer housing is pushed down by a user in relation to the inner housing, the inner and outer housing engage each other; otherwise the engaging elements of the inner and outer housing remain disengaged, which allows rotation of the outer housing in relation to the inner housing when the device is secured to an NFC. Features that allow transitioning between engaged and disengaged positions include springs or biasing or resilient elements or materials. Advantageously, the compressible cleansing matrix can serve as a spring to push the outer housing into a neutral position (disengaged from the engaging element(s) of the inner housing) such that a user can rotate the outer housing in relation to the inner housing (and NFC to which the device of the invention is attached). In other embodiments, when the device is attached to a needlefree connector and grasped by a user to supply adequate force to attach or detach the device to or from an NFC (i.e., adequate attachment or detachment force), the inner and outer housings adopt an engaged configuration relative to each other such that they can rotate in unison.

The outer housing advantageously includes a preferably cylindrical main cavity designed to receive and retain the inner housing using one or more features or elements configured for retention of the inner housing. The outer housing also includes one or more features or elements configured to engage with and disengage from complementary features or elements on the inner housing, preferably on the upper surface of the inner housing that allow the outer housing to be rotated in relation to the inner housing if and when desired. The main cavity is formed by a curved outer sidewall that in some embodiments is joined to a top portion of the outer housing about its periphery and which also preferably has a concentric central matrix well or matrix attachment region to or with which the compressible cleansing matrix is attached or otherwise associated, although in some embodiments some degree of eccentricity between the matrix well and central rotational axis of the outer housing may be desired. In some embodiments, the outer housing is formed by a sidewall that is tapered and/or has one or more steps.

In some preferred embodiments, the inner surface of the top of the outer housing includes one or more inner housing engaging elements or structures (e.g., teeth) designed to releasably engage complementary structures in the outer housing-engaging region on the top of the first inner housing. Engagement of the outer housing’s inner housing engaging structure(s) with those in the outer housing-engaging region of the inner housing allow a user to rotate the outer housing and inner housing in unison, for example, as a cleansing and capping ’s inner housing is screwed onto the threaded portion of a needlefree connector to be cleansed and/or capped. Once the device is releasably secured to a needlefree connector via the inner housing, the outer housing’s inner housing engaging elements or structure(s) can be (or are) disengaged from the outer housing-engaging elements of the inner housing, for example, by the biasing action or resilience of a the compressible cleansing matrix, thereby allowing a user to rotate the outer housing about its central axis in relation to the inner housing in order to scrub surfaces of the NFC in contact with the compressible cleansing matrix. Features such as an inner housing having an opening in its top to allow a compressible cleansing matrix attached to the inner surface of the top of the outer housing to extend into the central bore of the inner housing so that it can contact surfaces of a needlefree connector upon connection of the former to the latter are also envisioned.

In some of embodiments, the outer housing may include one or more vents to allow fluid and/or air from inside the device to escape as the cleansing and capping device is secured to a needlefree connector, while in other embodiments, no vent(s) is(are) provided. In embodiments with one or more vents, a membrane, filter, or other permeable or semi-permeable barrier may be employed to allow a unidirectional or bidirectional flow of air, gas, or vapor through the vent(s) but prevent the movement of microorganisms (e.g., bacteria, fungi, viruses, etc.) into the cleansing and capping device of the invention.

In certain preferred embodiments, the outer surface of the outer housing of a cleansing and capping device according to the invention includes one or more grip-enhancing structures (e.g., a plurality of vertical ridges, ribs, bumps, and/or the like) or coatings. Such grip-enhancing structures or coatings facilitate a user’s grasp of a particular housing of a cleansing and capping device between her/his fingers, which can be helpful not only during attachment and removal of a needlefree connector from the capping cleansing device, but also during the cleansing process, where, for example, in many embodiments the user rotates the outer housing in relation to the first inner housing in order to scrub and thereby clean or cleanse the surface(s) of the attached needlefree connector with the compressible cleansing matrix of the device.

In some preferred embodiments, the devices of the invention include one or more elements or features arrayed on facing surfaces of the inner and outer housings that allow a user to sense that the upper housing is rotating in relation to the inner housing in order to provide cleansing action on the valve surface of the NFC to which the device is attached. Such sensory feedback can include one or more of auditory, tactile, and/or visual stimuli generated from the device by rotation of one housing in relation to another of the device’s housings. Tactile feedback upon tight attachment of a device of the invention to a needlefree connector is also advantageous and can be realized by the devices of the invention.

The devices of the invention also include an inner housing disposed in the outer housing that allows the device to be attached and detached from the threaded valve portion of a needlefree connector. Each inner housing has a wall that forms a central, interior (preferably cylindrical) bore that extends between oppositely disposed upper and lower openings, respectively. The interior wall of the central bore nearer the second (lower) opening includes one or more threads or thread-engaging tabs preferably two helical threads disposed opposite to each other. The thread-engaging tab(s) or threads are configured to engage a complementary threaded region on the exterior surface of, for example, a needlefree medical valve. This allows the cleansing and capping device to be securely threaded onto the targeted threaded portion of a needlefree connector for cleansing and, if desired, capping that portion of the needlefree connector. The exterior surface of the inner housing includes one or more structures that allow it to mechanically engage and disengage complementary structures disposed on an inner surface of the outer housing as well as one or more structures that allow the inner housing to be rotatably retained in the outer housing.

In many embodiments, the inner housing’s exterior surface includes an outer housing-engaging region that includes one or more structures such as spaced protrusions (e.g., teeth) that allow the inner housing to mechanically engage and disengage complementary engaging structures on an interior surface of the outer housing. Preferred embodiments of outer housing-engaging structures include alternating teeth and grooves (or channels) circumferentially arrayed about the upper surface of the central bore of the inner housing, which teeth and grooves are complementary to one or more spaced engaging structures (e.g., teeth) disposed on the inner surface of the top of the outer housing in the region between the outer housing’s sidewall and wall (or other structure(s)) that defines the matrix well or region protruding from the interior surface of the top of the outer housing. wall. As will be appreciated, in configurations that include teeth and channels, the “teeth” can be raised protrusions and the “channels” can be the spaces or gaps between the raised protrusions.

The inner housing also includes a flexible compressible region or biasing element (e.g., a spring) adapted to engage the inner housing-retaining element on the interior surface of the sidewall of the outer housing. In preferred embodiments, the flexible compressible region or biasing element protrudes outward from the outer surface of the inner housing, such that its distal region is spaced from the wall that forms the portion of the inner housing that includes the central bore, with the space between the biasing element (or “skirt”) being a gap, the spans of which can change. The proximal region of the biasing element defines where that element extends from the inner housing wall and is opposite from the distal end or region of the biasing element. Preferably, an inner housing’s flexible compressible region or biasing element includes an outer housing retaining element configured to interface with the inner housing retaining element of the outer housing. In some preferred embodiments, the flexible compressible region or biasing element of the inner housing is configured as a flexible circumferential flange (or spaced flange elements) or skirt. Advantageously, the circumferential flange or skirt (or spaced flange or skirt elements), which flares downward and away from the wall of the inner housing that defines the central bore, is sized to be slightly wider in its lower area than the width of the inner housing retaining element of the outer housing so that, in an inner and outer housing subassembly, it is biased so as to provide a slight force against the outer housing’s inner housing retaining element, provided that such force will not (i) appreciably interfere with vertical movement of the outer housing in relation to the inner housing in response to upward force applied by the compressible cleansing element against the outer housing following attachment of the device of the invention to an NFC (which compresses the cleansing matrix), after a user releases the attachment force used to attach the device to an NFC, and (ii) appreciably inhibit rotation of the outer housing in relation to the inner housing during a cleansing operation. As will be appreciated, when the outer housing’s inner housing retaining element and outer housing retaining element of the inner housing are continuous (i.e., uninterrupted or not segmented) about their circumference, they can provide a seal, if desired.

Each device of the invention also includes an outer housing in operable association with the inner housing. An outer housing typically includes a main cavity formed by a curved outer wall that is joined to a top portion, preferably about the top portion’s periphery, and a preferably concentric central matrix well that extends from the top’s inner surface into the main cavity. The wall forming the matrix well is spaced from the outer housing’s outer sidewall to form an inner housing receiving zone that can be accessed through an opening created by the gap between the outer housing’s sidewall and the matrix well wall. That zone can include inner housing engaging elements configured to engage complementary outer housing engaging elements atop the inner housing.

Engagement of inner and outer housings’ engaging or locking structure(s) with those in the outer housing-engaging region of the inner housing allows a user to rotate the inner and outer housings in unison, for example, as a device of the invention is screwed onto the threaded portion of a needlefree connector to be cleansed and/or capped. Once the device is releasably secured to a needlefree connector, the outer housing’s locking structure(s) can be disengaged from those in the outer housing-engaging region of the inner housing, thereby allowing a user to rotate the outer housing about its central axis in relation to the inner housing.

The cleansing and capping devices of the invention also include a compressible cleansing matrix in the outer housing’s matrix well. As will be appreciated, in many embodiments the compressible cleansing matrix can itself act as a resilient body or spring. The compressible cleansing matrix can be, for example, an open-cell foam. The cleansing matrix is preferably secured to an inner surface of the outer housing so as to limit or restrict its rotation independent of the outer housing during outer housing rotation. The compressible cleansing matrix is configured to contact and cleanse one or more surfaces of a needlefree connector that contacts the matrix upon a needlefree connector’s association with a cleansing and capping device of the invention. The compressible cleansing matrix attached to or otherwise associated with the outer housing can be axially compressed (i.e., compressed along the central axis of the outer housing’s matrix well) upon insertion of a needlefree connector into a cleansing and capping .

Because the needlefree connector surface(s) to be cleansed may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the compressible cleansing matrix preferably has sufficient mechanical integrity when compressed to allow its use to disrupt any biofilm that may be present on a surface of the needlefree connector that is contacted by the cleansing matrix. Disruption of biofilm can occur by rotating, twisting, or otherwise moving a then-compressed cleansing matrix in relation to the needlefree connector (e.g., a needlefree medical valve), for example, by rotating the outer housing (to which the compressible cleansing matrix is attached or otherwise associated or retained) in relation to the inner housing of the cleansing and capping device and the needlefree connector to which the inner housing is releasably attached. The resulting friction between the compressed cleansing matrix and surface of the needlefree connector disrupts the biofilm, thereby cleansing, and preferably sterilizing, the needlefree connector. Leaving the cleansing and capping device secured to (i.e., capping) the needlefree connector after such cleansing will limit, and preferably preclude biofilm regrowth and/or the microbial recolonization of cleansed surfaces (which remain in contact with the compressible cleansing matrix) of the needlefree connector.

In preferred embodiments, the compressible cleansing matrix includes one or more cleansing reagent species dispersed therein, preferably at the time the device is manufactured, although in some embodiments, the cleansing reagent may be dispersed into the matrix just prior to the matrix coming into contact with a needlefree connector. In embodiments of the latter sort, the cleansing reagent is preferably housed in the body of the cleansing and capping device in a reservoir configured to rupture upon association of a needlefree connector for cleansing. Such a reservoir can be disposed between the matrix and needlefree connector, or, more preferably, between the rotatable cap and compressible cleansing matrix. Preferred cleansing reagents include antimicrobial reagents such as isopropyl alcohol, chlorhexidine, and silver ions. In some embodiments, the cleansing and capping device of the invention will include a valve or opening to allow liquid in the cleansing reagent to evaporate.

As described, the central matrix well of the outer housing is adapted to receive the compressible cleansing matrix. The surface(s) of the central matrix well in contact with the matrix preferably includes one or more retaining structures to retain the compressible cleansing matrix so as to link its rotation or movement to that of the cap, particularly when the engaging structures of the cap and resilient inner body are disengaged so as to allow cap rotation during a needlefree connector cleansing procedure. Such retaining structures include ridges and other protrusions from the surface of central matrix well in contact with the compressible cleansing matrix. An adhesive can also be used to adhere that portion of the compressible cleansing matrix to a desired position in the matrix well.

In various embodiments, the outer surface of the outer housing of a cleansing and capping device according to the invention includes one or more grip-enhancing structures or coatings, e.g., a plurality of vertical ridges, ribs, bumps, or the like (including combinations of such structures and/or coatings). Such grip-enhancing structures or coatings facilitate a user’s grasp of the body of a cleansing and capping device between her/his fingers, which can be helpful not only during insertion and removal of a needlefree connector from the cleansing and capping device, but also during the cleansing process, where the user rotates thew outer housing in relation to the inner housing and NFC in order to scrub and thereby clean/cleanse the surface(s) of the inserted needlefree connector with the compressible cleansing matrix and cleansing reagent (e.g., 70% IPA).

The inner and outer housings of the devices of the invention can be made from any suitable material or combinations of different materials. Plastics are particularly preferred. The material(s) used to manufacture the outer housing may be the same or different as the material(s) used to produce the inner housing.

The inner and outer housings and their various components are preferably formed as a single, integral unit during manufacturing (e.g., by injection molding). The inner and outer housings can be manufactured by any suitable process, including extrusion, injection molding, and additive manufacturing (e.g., 3D printing). After manufacturing, the inner housing is operably attached to or connected with, for example, by insertion into, the outer housing to form a inner and outer housing subassembly ready to assemble into a complete cleansing and capping device of the invention. For securing the inner and outer housings together as a functional subassembly that can transition between engaged and disengaged configurations to provide for unitary or independent rotation of the outer housing in relation to the inner housing, any suitable retaining structure, or group of complementary structures, that provides for movement, i.e., rotation, of the outer housing in relation to the inner housing can be used. Such structures include attachment mechanisms such as “snap-fit” mechanisms where interacting parts are sufficiently flexible and have preferably tapered surfaces to facilitate assembly.

A compressible cleansing matrix can be positioned in the matrix well before or after an inner housing and outer housing are operably associated or assembled. In some embodiments, a suitable adhesive is used to securely adhere the compressible cleansing matrix, or, if the matrix comprises two or more parts, its various portions, to one or more inner surfaces of the second outer housing’s matrix well. In some embodiments, the surface of the matrix well that contacts the compressible matrix includes one or more structures (e.g., splines, ribs, posts, etc.) to assist in securely retaining the matrix in the well, thus ensuring that it moves in conjunction with the cap when the outer housing is rotated during a cleansing procedure.

In preferred embodiments, the cleansing and capping devices of the invention include a removable lid or seal attached to one or more housing to seal the device, thus separating the interior spaces and structures of the device from the external environment. Such a lid or seal prevents exposure of the device’s interior, including, for example, its inner (e.g., lower) housing and compressible cleansing matrix, to the environment until the seal is removed, typically by a healthcare worker just prior to her/his use of the cleansing and capping device to cap and then, if desired, to clean/cleanse the needlefree connector (e.g., needlefree medical valve) to which it is connected. In preferred embodiments, such cleansing substantially disrupts any biofilm that may exist on surfaces contacted by the compressible cleansing matrix. If desired, the cleansing and capping device can be left in place (typically after cleansing the needlefree connector attached thereto) in order to cap the needlefree connector until it is further accessed, thereby minimizing exposure of capped exterior surfaces of the connector to potential pathogen contamination (and biofilm formation) from the surrounding environment. Seals are typically installed during manufacture of a cleansing and capping device of the invention. In those embodiments where the cleansing and capping devices are sterilized during manufacture (e.g., by irradiation, exposure to ethylene oxide, etc.), seals are preferably applied prior to sterilization.

Other aspects of the invention concern methods of cleansing and/or capping needlefree connectors using a cleansing and capping device according to the invention. In many embodiments, such methods typically involve transitioning the inner and outer housings from an engaged to a disengaged configuration after the device has been connected to a needlefree connector as to allow the outer housing to spin or rotate in relation to the inner housing. In embodiments where the outer housing is downwardly compressed to engage the inner housing, such transitioning facilitates contact between the device’s compressible cleansing matrix and the associated surface(s) of the needlefree connector. Spinning or rotation of the outer housing in relation to the inner housing, and the associated surface(s) of the needlefree connector, allow those surfaces to be scrubbed, thereby cleansing them. Preferably, such cleansing methods provide for the disruption of any biofilm present on the surface(s) of the needlefree connector associated the cleansing and capping . And in those embodiments where the compressible cleansing matrix contains one or more antimicrobial reagents, microbes and pathogens present in such biofilm and/on on such surface(s) are destroyed or rendered nonviable.

Herein, the compressible cleansing matrix of a cleansing and capping device of the invention comprises one or more cleansing reagent species dispersed in a substrate. The cleansing matrix substrate can be any substance that can conform, mold, or compress in a manner that enables the effective friction-based cleansing of the site or portion of the needlefree connector to be cleansed, including the top surface of the site, side surface, and any threads or grooves, if present, and provide the cleansing reagent at least at a surface level. Examples of the compressible cleansing matrix include cotton, open or closed cell foam such as polyethylene foam, or other substance that can hold or carry the cleansing reagent.

In some embodiments, the cleansing reagent species is(are) dispersed in or otherwise combined with the compressible cleansing matrix during the process used to manufacture the cleansing and capping , while in other embodiments, the device is configured such that the cleansing reagent(s) is(are) released for dispersion into the compressible cleansing matrix post-manufacture, but when or prior to the time the matrix is brought into contact with the needlefree connector to be cleansed. The cleansing reagent can be any chemical, substance, or material that cleans the site of bacterial or even viral microorganisms, biofilm, etc., or any carrier that contains such chemical, substance or material. Examples of cleansing reagents include isopropyl alcohol, chlorhexidine, chlorhexidine digluconate, povidone-iodine, hydrogen peroxide, soap, and hydrochloric acid, silver ions and salts (e.g., silver acetate, silver lactate, silver nitrate, etc.), etc.

In accordance with the invention, a cleansing reagent comprises an active ingredient capable of cleansing a surface of a needlefree connector. Any active ingredient that can be used effectively to rapidly cleanse a medical fitting or medical line connector (e.g., a needlefree connector) can be adapted for use in practicing the invention, and are generally classified as antibacterial and/or antifungal reagents, antiseptic or antimicrobial reagents, wide spectrum disinfectants, and/or parasiticides, as well as combinations of such reagents. Particularly preferred are biocompatible cleansing reagents, as the devices of the invention are intended for human and/or veterinary use, including alcohols, antibiotics, oxidizing reagents, and metal salts. Representative examples of such active ingredients include bleach, chlorhexidine, ethanol, isopropyl alcohol, hydrogen peroxide, sodium hydroxide, and an iodophor dissolved or otherwise dispersed in a suitable solution, suspension, or emulsion. Other active ingredients having suitable cleansing effects can also be used. These include alcohols (e.g., ethanol, benzyl alcohol, isopropyl alcohol, phenoxyethanol, phenethyl alcohol, etc.); antibiotics (e.g., aminoglycosides, such as amikacin, apramycin, gentamicin, kanamycin, neomycin, netilmicin, paromomycin, rhodostreptomycin, streptomycin, and tobramycin; bacitracin; chloramphenicol; erythromycin; minocycline/rifampin; tetracycline; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; etc.), quaternary ammonium chlorides; quaternary ammonium carbonates; benzalkonium chloride; chlorinated phenols; fatty acid monoesters of glycerin and propylene glycol; iodine; iodine containing compounds, such as 3-iodo-2-propynyl butyl carbamate (IPBC); iodophors, such as povidone-iodine (Betadine 100%, which contains providine iodine as the active ingredient); hydantoins, such as dimethylhydantoin and halogenated hydantoins; isothiazolinones; parabens, such as methylparaben, ethylparaben, and propylparaben; chloroxylenol; chlorhexidine and its salts; chlorhexidine/silver-sulfadiazine; chlorhexidine acetate; chlorhexidine gluconate (e.g., Hibiclens); chlorhexidine hydrochloride; chlorhexidine sulfate; benzoic acid and salts thereof; benzalkonium chloride; benzethonium chloride; methylbenzethonium chloride; chlorobutanol; sorbic acid and salts thereof; imidazole antifungals (e.g., miconazole); butocouazole nitrate; mafenide acetate; nitrofurazone; nitromersol; triclocarban; phenylmercuric nitrate or acetate (0.002%); chlorocresol; chlorbutol; clindamycin; CAE (Anjinomoto Co., Inc., containing DL-pyrrolidone carboxylic acid salt of L-cocoyl arginine ethyl ester); cetylpyridinium chloride (CPC) at 0.2%, 0.02%, and 0.002% concentrations; 9.8% isopropyl alcohol; 1% ZnEDTA; mupirocin; and polymyxin (polymyxin b sulfate-bacitracin). Additionally, other useful compounds and compositions include Miconazole, Econazole, Ketoconazole, Oxiconizole, Haloprogin, Clotrimazole, butenafine HCl, Naftifine, Rifampicin, Terbinafine, Ciclopirox, Tolnaftate, Lindane, Lamisil, Fluconazole, Amphotericin B, Ciprofloxecin, Octenidine, Triclosan (2,4,4'-trichloro-2'-hydroxydiphenyl ether), Microban (5-chloro-2phenol (2,4 dichlorophenoxy). Useful metals include silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine. Cleansing reagents are often compositions that comprise the desired active ingredient(s) in admixture with other ingredients, such as carriers and liquid solvents.

The particular active ingredient(s) selected as a cleansing reagent for a given application will be compatible with the compressible cleansing matrix and material(s) used to form the housings and other components of the particular device. In some embodiments, the cleansing reagent is dispersed in the compressible cleansing matrix after the matrix is formed. For example, a cleansing reagent can be dispersed by saturating or supersaturating a compressible cleansing matrix during manufacture of the device, preferably before it is sealed. In other embodiments, the cleansing reagent can be dispersed during the process used to manufacture the compressible cleansing matrix. As will be appreciated, the materials used to prepare the cleansing reagent should be compatible with the constituent or constituents that comprise the compressible cleansing matrix such that the substrate does not appreciably degrade or otherwise suffer loss of structural integrity prior to being used to cleanse a needlefree connector (e.g., a needlefree medical valve). Similarly, the cleansing reagent should be biocompatible, such that it will not harm a patient in the event of contact or should some amount of the cleansing reagent be admitted into the fluid-carrying portion of a needlefree medical valve, as well as with materials used to form needlefree medical valves (or other needlefree connector).

In preferred embodiments, the material used to form the compressible cleansing matrix is any suitable absorbent, compliant, pliable, resilient, fibrous, or porous material, or combination of materials, than can be wetted and/or impregnated with a cleansing reagent and which can easily and readily adapt to complex surface contours (e.g., luer threads, concave and convex surfaces, flanges, etc.) likely to be engaged upon contact with, for example, a needlefree medical valve to be cleansed. Such materials include those that are synthetic or naturally occurring, and they may be of homogeneous or heterogeneous composition. Preferred synthetic materials include fibrous, foam (e.g., a felted foam), and gel compositions, particularly those having directionally oriented natural or synthetic fibers, or combinations thereof. Preferred naturally occurring materials useful as substrates include fibrous naturally occurring materials, including plant-derived materials such as cotton and paper products, as well as animal-based fiber products such as wool. Other preferred natural materials are sponges.

As will be appreciated, in order to achieve the desired cleansing effect, a compressible cleansing matrix, or the component part(s) thereof designed to contact a needlefree connector such as a needlefree medical valve, preferably are made of a material (or combination of materials) that allow the cleansing element to thoroughly cleanse surfaces of needlefree connectors such as needlefree valves or luer access devices, particularly those surfaces that are exposed to air and thus are at risk for contamination with infectious or pathogenic reagents, and biofilms containing the same, and are also intended to form part of the fluid flow path for fluids to be introduced into a patient, for example, IV solutions, medications, blood and blood products, etc.

Preferably, the material used to produce the compressible cleansing matrix should be sufficiently compliant to allow the compressible cleansing matrix to deform under the pressures experienced during normal use in order to allow it to conform to the external structures present on the surface(s) of the needlefree connector to be cleansed. This assures intimate, cleansing contact between the compressible cleansing matrix and at least those exposed surfaces of, for instance, a needlefree connector designed to come into contact with fluid entering the valve, such as IV fluids. In addition, the compressible cleansing matrix preferably allows for the retention of a liquid cleansing reagent, for example, in capillary spaces, in the void volume of foams, sponges, etc. The compressible cleansing matrix may also be engineered such that it includes cleansing reagents such as silver ions and/or other suitable materials.

Preferred natural materials from which compressible cleansing matrices can be formed include those derived from cotton and naturally occurring sponges. As those in the art appreciate, processed cotton fibers are composed almost entirely of the natural polymer cellulose. In such fibers, 20-30 layers of cellulose are coiled into a series of spring configurations, which makes the fibers absorbent and gives them a high degree of durability and strength. For example, woven cotton sheets, as are often used in the manufacture of sterile cleansing pads that are then saturated with a 70% isopropyl alcohol (IPA) solution, can be used as substrates for cleansing elements according to the invention. Any suitable configuration may be used. For example, a woven cotton sheet can be cut into numerous similarly sized pieces, each of which can be used as a substrate. In many embodiments, after attachment to the inside surface of a layer of the container (e.g., through the use of an adhesive, double-sided, tape, etc.), the matrix is ready for the addition of a suitable cleansing reagent. Alternatively, cotton fibers can be spun onto the inside surface of the cap. Other fibers, be they naturally occurring, synthetic, or combinations of natural and synthetic materials, having similar properties can also readily be adapted for use as compressible cleansing matrices.

Another class of materials for compressible cleansing matrix fabrication is directionally oriented fibrous materials. These include, without limitation, materials comprised of cellulose fibers, glass fibers, and polyester fibers, as well as materials comprised of combinations of two of more of these and/or other materials. Such bonded synthetic fibers use capillary action to precisely absorb, retain, transfer, and/or release liquids or vapor in desired amounts. A broad range of synthetic polymers can be used to form the fibers, and, if desired, they may be treated for functional purposes, for example, to contain a cleansing reagent dispersed therein, to provide a vapor barrier or other coating over a portion of the product’s surface, etc. The geometric shape of these materials can also be customized for particular applications, thereby permitting easy integration into substrate configurations having the desired device thickness, widths, length, diameter, etc.

Other representative classes of materials suitable for use as compressible cleansing matrices include gel-forming polymers and foams such as agarose, agar, polyacrylamide, and other synthetic porous materials that can be formed into layers, sheets, columns, or other shapes compatible with practicing the invention. Representative gelatinous materials include hydrogels (i.e., cross-linked polymers that absorb and hold water), particularly those made from agarose, (2-hydroxyethyl)methacrylate and its derivatives, and synthetic carbohydrate acrylamides.

Still other classes of materials include porous polymer sponges. Such sponges can be formed from any suitable material, including polyethylene, polypropylene, olytetrafluoroethylene, polyvinylidine difluoride, polynitrile, and polystyrene. Many such porous polymer sponges are commercially available in a wide variety of shapes, pore density and size, etc. Additionally, polymer sponges can be made by polymerizing appropriate monomers according to conventional foam forming techniques. In general, sponges have an open pore structure to allow movement of a solvent such as a liquid cleansing reagent. The sponge surface should include open pores to provide entry of liquid cleansing reagents (e.g., alcohol, iodine-containing solutions, etc.), and, as with other materials used to form matrices, the particular material chosen is preferably inert, i.e., not reactive with components of the cleansing reagent, the body of the cleansing and capping , or the materials used to produce needlefree connectors such as needlefree medical valves.

Surgical foams are another preferred class of materials that can be used to make compressible cleansing matrices. The materials can be natural or synthetic, as desired. Suitable foams include rubber latex, polyurethane, polyethylene and vinyl foams. Preferably, such foams are made from any suitable biocompatible polymer, for example, polyvinyl alcohol (PVA) or polyurethane. One preferred foam material is Microbisan™, a hydrophilic polyurethane foam that is impregnated with silver ions (Lendell Manufacturing, St. Charles, MI). Preferably, such foams are highly absorbent and thus suitable for use with liquid cleansing reagents. In other embodiments, the material used to form the foam is well-suited for dispersion of a dry cleansing reagent, such as silver ions. Again, it is preferred that foam materials, if used to as a substrate, be inert. Also, they are preferably sufficiently flexible to conform to the variety of different shapes and surface configurations (e.g., double seal fluid access points, luer threads, etc.) encountered in the field given the multitude of medical valve shapes, sizes, and configurations. In this way sufficient contact between the cleansing surface(s) of the cleansing and capping device and the surface(s) of the needlefree connector to be cleansed can be ensured. Another advantage of some synthetic foams (as well as certain other polymeric materials from which substrates may be formed) is that they can easily be injected in a desired volume into a shell or housing during manufacture, after which they expand to assume the desired substrate size, density, porosity, etc.

Furthermore, compressible cleansing matrices can include chemicals to indicate a functional change therein, for example, by using a color change to signal a change from a wet to a dry state, or, alternatively, that the matrix material has been properly wetted with a liquid cleansing reagent dispersed into the substrate by a health care worker just prior to use, as opposed to during manufacture of the device. Thus, depending on the system used, a color change in the matrix could be used to indicate that the cleansing reagent in the compressible cleansing matrix has evaporated prior to use and thus the particular cleansing device should not be used, perhaps due to a leak in the cleansing and capping device’s storage container. Alternatively, when, for example, a colored liquid cleansing reagent is used, the user can visually confirm dispersion of the reagent in the matrix by assessing whether the colored cleansing reagent is dispersed throughout the matrix. When colored cleansing reagents are used, it is preferred that the material(s) use to make the resilient inner body and/or cap of the cleansing and capping device be clear or translucent, or include Capping and cleansing devices of the invention and their constituent parts (e.g., inner and outer housings, compressible cleansing matrix, disinfectant, sealing ring (if present), lid/seal, etc.) can be made from any suitable material(s) and assembled using any suitable process.

Preferably, at least a portion of the outer surface(s) of a cleansing and capping device of the invention (e.g., the outer housing.) that is intended for grasping by a user has a non-slip surface, i.e., one having a high coefficient of friction so that when the outer portion of a cleansing and capping device is held in a user’s fingers and positioned to cleanse a needlefree connector so that the housing(s) can be rotated in relation to the inner housing and needlefree connector with minimal or no slippage between the device and the user’s fingers (gloved or ungloved). Examples of such non-slip (or high friction) surfaces include those having ridges, valleys, dimples, bumps, or other features designed to enhance friction, as well as combinations of two or more of such features. Such features can be introduced into a device’s outer surface(s) as part of the manufacturing process. Alternatively, a non-slip coating can be applied to one or more of the outer surfaces of outer housing.

In general, the cleansing and capping devices of the invention are provided to users in a sealed, sterile manner. If desired, labeling information, logos, artwork, manufacturing, and/or regulatory data (e.g., lot number, expiration or “use by” dates, etc.) may also be printed or otherwise applied to individual cleansing and capping devices. In addition, information such as a bar or QR code (to allow use of the device to tracked, for example) may also be included on individual cleansing and capping devices.

As will be appreciated, cleansing devices may be packaged individually or in groups of two or more units as kits, which can further include instructions for use of the cleansing and capping device(s), as well as other information, logos, artwork, manufacturing, and/or regulatory data.

In preferred embodiments, packaged cleansing and capping devices are sterilized using a suitable process, such as irradiation. In a particularly preferred practice, the cleansing and capping devices of the invention are sterilized as part of the manufacturing process. Here, “sterilization” refers to any process that effectively kills or eliminates transmissible reagents, e.g., bacteria, viruses, fungi, prions, spores, etc. that may be present in any component of a device according to the invention. In preferred embodiments, sterilization can be achieved by heating, chemical treatment, irradiation, and other processes. Indeed, any sterilization process compatible with the materials used to make the cleansing and capping device can be employed. A particularly preferred sterilization process is an irradiation process. Such processes include irradiation with x-rays, gamma rays, or subatomic particles (e.g., an electron beam). In general, when a sterilization process is used in the context of the invention, the process is employed on a cleansing article after it has been sealed and/or packaged. Chemical sterilization processes can also be used, for example, sterilization using ethylene oxide (EtO).

### Representative Embodiments

To further illustrate and describe a preferred, representative embodiment of the invention, the reader is directed to the appended drawing, FIG. 1, which illustrates a particularly preferred embodiment of a cleansing and capping device (1) according to the invention, as described above.

FIG. 1 shows a device according to the invention that includes an inner housing (7) disposed in the cavity (31) of an outer housing (4), a compressible cleansing matrix (10) with a liquid disinfectant disposed therein, and a removable seal (13). Such devices are intended for single use, are preferably sterilized, and can be configured as individually sealed units or arrayed as a plurality of units (e.g., 3-10 or more) sealed using a single piece of seal or lid material, which can advantageously be hung, for example, from an IV pole at a patient’s bedside in order to be readily accessible to a nurse providing, for example, IV therapy to a patient via an NFC in an IV set connected to the patient, which NFC is to be cleansed and capped until a subsequent fluid access is made through the same connector.

In a device as shown in FIG. 1, the inner housing (7) is configured for secure, removable attachment to a threaded valve portion (85) of a needlefree connector (82), allowing the entire device to be removable yet securely attached to an NFC. Advantageously, when a user attaches the device (1) tightly to an NFC (82), the user experiences a “hard stop” sensation, indicating that further application of attachment force is not required.

The inner housing (7) includes a central bore (40) having oppositely disposed first (e.g., top) and second (e.g., bottom) openings. In this embodiment, the first and second openings are of different sizes, with the top opening (67) being larger than the bottom opening (70). Furthermore, in the illustrated embodiment, the central bore has different diameter over its length. Here, the upper portion (43) of the central bore (40) has a larger internal diameter than the central bore’s thread-containing lower portion (46), resulting in a step (49) or similar structure in the interior surface of the central bore at the transition between its upper and lower regions. The sidewall (34) of the upper central bore region advantageously also preferably has a height (or length)(including the outer housing (4) engaging elements) that is greater than the height (or length ) of the outer housing’s matrix well (16) wall (19).

As shown, the inner housing (7) is configured to allow the device to be screwed onto and unscrewed from the threaded valve portion (85) of an NFC (82) while at the same allowing the compressible cleansing matrix (10) attached to (or carried by) the outer housing (4) to protrude into the central bore (40) through the first (upper) opening so as to allow contact between the compressible cleansing matrix and the NFC’s valve surface when the device is screwed onto the threaded valve portion of an NFC. Advantageously, this can be accomplished by including threads (76) (or thread tabs) on the interior surface of at least the lower central bore region.

The inner housing (7) further includes a downwardly projecting, flexible, circumferential biasing element (64) or “skirt” region that protrudes from its outer surface, an outer housing (4) retaining element near the bottom (lower portion) exterior or outer surface of the skirt region of the inner housing, and one or more outer housing engaging elements disposed on an upper surface of the inner housing. The biasing element (64) is designed so that it can be compressed from a first (retaining) position to a second (or “compressed” or “assembly”) position during assembly of the inner housing (7) into the cavity (31) of the outer housing (4). Such movement of the biasing element is inward, toward the outer wall of the lower region of the central bore (40), as happens during assembly of the inner and outer housings into a rotatable subassembly. Such inward movement causes the compression gap (61) between the biasing element (skirt, 64) and adjacent portion of the central bore to decrease. Upon completing assembly of the rotatable subassembly, the biasing element (64) of the inner housing (7) rebounds into a partially compressed third position, advantageously intermediate between the first and second positions, with the compression gap (61) substantially returning to its original size or being slightly smaller than when in the first (free) position.

In the illustrated embodiment, the outer housing retaining element (55) of the inner housing (4) is a channel or slot disposed on a lower portion of the outer surface of the biasing element (64). This outer housing retaining element (55) is configured to engage a complementary inner housing retaining element (58) that extends from an inner (or interior) surface of the outer housing’s sidewall (34). The inner housing’s outer housing retaining element (55) is also sized to allow the outer housing to vertically move in relation to the inner housing, for example, as occurs when the outer housing (4) is pushed upward in relation to the inner housing (7) by the compressible cleaning matrix (10) into a neutral position useful for scrubbing an NFC after a user attaches the device (1) to a threaded valve region (85) on an NFC (82). To provide for such vertical movement (up and down) of the outer housing (4) in relation to the inner housing (7), the height (or length) of the inner housing’s outer housing retaining element (55) is advantageously about 2-(10) times or more the height of the outer housing’s inner housing retaining element (58). The inner housing (7) also includes one or more outer housing engaging elements (52), which are preferably disposed on an upper surface of the inner housing (4). Advantageously, in the depicted embodiment, such outer housing engaging elements (52) are configured as “teeth” designed to engage complementary structured inner housing engaging elements (22) disposed on the interior surface (73) of top (72) of the outer housing (4) in the region between the outer housing’s sidewall and matrix well (16) wall (19).

In the embodiment shown in FIGS. Fig. (1), the overall height of the inner housing (7) is less than the depth of the cavity (31) of the outer housing (4). In this way, the inner housing (7) is thus recessed inside the outer housing, even when the engaging elements of the inner and outer housings are disengaged (i.e., the outer housing (4) is in a “neutral” position, which allows it to be rotated in relation to the inner housing (7), particularly when the device (1) is removably attached to the threaded valve region (85) of an NFC(82)).

The outer housing (4) of the device shown in FIG. 1 is rotatably connected to the inner housing (7). The outer housing (4) comprises a cavity (31) in which the inner housing (7) is rotatably disposed and retained. The outer housing (4) is configured to not only to retain, via complementary retention elements (55, 58) on facing surfaces of the inner and outer housings (7, 4, respectively), the inner housing (7) in the cavity (31), but also to engage and disengage from the outer housing engaging elements (52) of the inner housing (7). This allows the outer housing (4) to (i) retain the inner housing (7) yet still be rotated independently of the inner housing in both a clockwise direction and a counter-clockwise direction by a user when complementary engaging elements on the inner and outer housings are not engaged and (ii) retain the inner housing (7) and be rotated in unison with the inner housing (7) by the user when the outer housing (4) and the inner housing (4) are engaged via their complementary engaging elements (22, 52, respectively).

In this embodiment, the outer housing (4) further comprises a matrix well (16) that extends into the cavity (31) from the inner surface (72) of the2 (73) of the outer housing (4) to securely retain a compressible cleansing matrix (10). As shown, the inner surface of the matrix well can advantageously include one or more matrix retention splines (28) which serve to help retain the compressible cleansing matrix in the matrix well and also to help translate rotational force to the compressible cleansing matrix (10) when a user performs a scrubbing operation using the device (1) to cleanse an NFC (82) to which it has been removably secured.

As described, the device shown in FIG. 1 also includes a compressible cleansing matrix (10) attached to the outer housing (4) that protrudes into the inner housing (7) and can rotate with the outer housing (4) in relation to the inner housing (7), particularly during user-driven cleansing operations. Advantageously, a liquid disinfectant, for example, a solution that is about 70% isopropyl alcohol and water (a 70% IPA solution) is present, advantageously in a volume that at least saturates the compressible cleansing matrix (10). If desired, the liquid disinfectant can further include chlorhexidine, for example, chlorhexidine gluconate.

In the device shown in FIG. 1, the compressible cleansing matrix (10) contacts at least the valve surface of the threaded valve portion (85) of an NFC (82) when the device is threaded onto the NFC. In some cases, the compressible cleansing matrix (10) can be configured to also contact one or more additional surfaces of the threaded valve portion (85) of an NFC when the cleansing and capping device (1) is removably attached thereto.

In the device shown in FIG. 1, the retaining elements (55, 58) of the inner and outer housings (7, 4, respectively) may also act a fluid seal between the housings, particularly if the retaining elements include facing surfaces configured to provide a fluid seal. Such sealing, however, is an optional, not essential feature, of the device.

In the device shown in FIG. 1, the device (1) further includes a removable lid or seal (13) to seal device from an external environment and prevent loss of the liquid disinfectant disposed in the device (1). Such a lid or seal (13) can advantageously be a peelable seal of any suitable material (or combination of materials).

FIG. 2 shows a device analogous to the device depicted in FIG. 1, the differences being that the inner housing retaining element (90) of the outer housing (4) of the device shown in FIG. 2 is an elongated channel, groove, or slot disposed on the interior surface of the lower portion of the sidewall (34) of the outer housing (4). This inner housing retaining element (90) is configured to engage a complementary outer housing retaining element (91) disposed on the lower portion of the outer surface of the biasing element (64) (skirt) of the inner housing (7). The outer housing’s inner housing retaining element (90) is also sized to allow the outer housing (4) to vertically move in relation to the inner housing (7), for example, as occurs when the outer housing is pushed upward by the compressible cleaning matrix into a neutral position useful for scrubbing an NFC after a user attaches the device to a valve region on an NFC. As is also the case with the device shown in FIG. 1, a user can push the device’s outer housing (4) downward (axially, or toward the NFC to which the device is (or is to be) attached) in relation to the inner housing (7) in order to attach or remove it from the NFC. To provide for such vertical movement (up and down) of the outer housing in relation to the inner housing, the height (or length) of the outer housing’s inner housing retaining element (90) is advantageously about 2-(10) times or more the height of the inner housing’s outer housing retaining element (91), which in the embodiment depicted in FIG. 2 is a raised, substantially hemispherically shaped circumferential band or ridge.

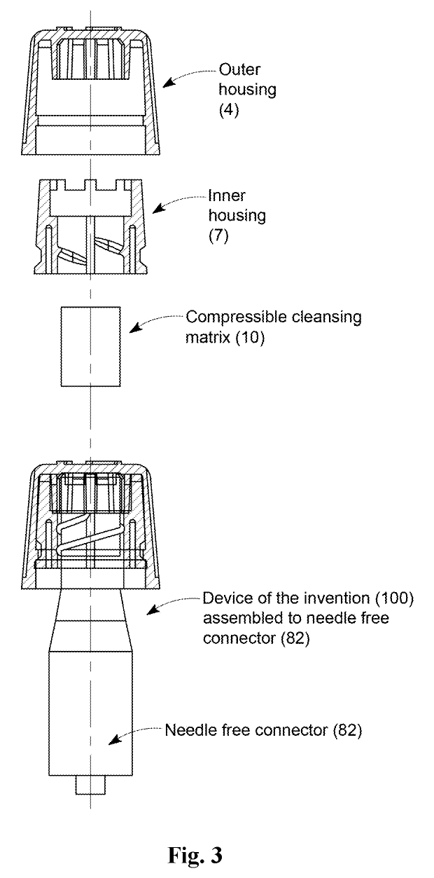

FIG. 3 illustrates a protected NFC assembly (100) that includes an unsealed device of the invention (1) as depicted in FIG. 1 attached to a needlefree connector (82) such that the compressible cleansing matrix (10) is compressed due to the inner and outer housings (7, 4) being in an engaged configuration (i.e., the engaging elements of the inner and outer housings being engaged) as (or after) the device is threaded on to the threaded valve portion (85) of an NFC (82). A user applying attachment or detachment force to the outer housing (4) to produce such engagement is not shown. As will be appreciated, in the absence of an unopposed engaging force (as would occur, for example, when a user lets go of the device after securely attaching it to an NFC), the compressible cleansing matrix (10) will exert a disengaging force that will cause the outer housing (4) to move vertically in relation to the inner housing (7) (which removably attaches the device (1) to the NFC (82)). This will result in the engaging elements of the inner and outer housings becoming disengaged, putting the device in a “neutral” position where a user can rotate the outer housing (4) independently of the inner housing (7), as occurs during performance of a cleansing operation to clean and disinfect the NFC’s valve surface.

As those in the art will also appreciate, the invention envisions cleansing and capping devices that include alternative or additional structures that allow the inner and outer housings of a device according to the invention to engage or disengage, for example, to allow attachment or detachment from a needlefree connector (82) (e.g., for engagement or disengagement) or to provide for “active” scrubbing (e.g., by independently rotating the outer housing (4) in relation to the inner housing 7). For example, and without limitation, in some embodiments, instead of the complementary engaging element(s) of the inner and outer housings being disposed on the upper surface of inner housing and interior surface (73) of the top o(72) f the outer housing, respectively, the inner housing engaging element(s) of the outer housing may advantageously be disposed on the bottom (lower) surface of the matrix well and the interior, central bore-facing surface of a step (49) or transition in the sidewall (34) that forms the upper and lower portions of the central bore, respectively.

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

What is claimed is:

1. A cleansing and capping device for a threaded valve portion of a needlefree connector, comprising:
(a) an inner housing configured for secure, removable attachment to a threaded valve portion of a needlefree connector, the inner housing a threaded central bore that comprises oppositely disposed first and second openings and is configured to allow the device to be screwed onto and unscrewed from the threaded valve portion of the needlefree connector and into which a compressible cleansing matrix attached to an outer housing protrudes into the central bore to allow contact with a valve surface of the threaded valve portion of the needlefree connector when the device is screwed onto the threaded valve portion of the needlefree connector, wherein the inner housing further comprises (A) a downwardly projecting, flexible biasing element protruding from the inner housing’s outer surface that can be compressed from a first position to a second position toward an outer wall of the central bore during assembly of the inner and outer housings into a rotatable subassembly, wherein upon assembly of the rotatable subassembly, the biasing element of the inner housing rebounds into substantially the first position, (B) an outer housing retaining element disposed in an outer surface of the biasing element, wherein the outer housing retaining element is configured to engage a complementary inner housing retaining element that extends from an inner surface of the outer housing’s sidewall, and is sized to allow the outer housing to vertically move in relation to the inner housing, and (C) an outer housing engaging element disposed on an upper surface of the inner housing;
(b) an outer housing rotatably connected to the inner housing, wherein the outer housing comprises a cavity in which the inner housing is rotatably disposed, wherein the outer housing is configured to (A) retain, via complementary retention elements on facing surfaces of the inner and outer housings, the inner housing in the cavity, and (B) engage and disengage from the inner housing so as to allow the outer housing to (i) retain the inner housing and be rotated independently of the inner housing in both a clockwise direction and a counter-clockwise direction by a user when complementary engaging elements on the inner and outer housings are not engaged and (ii) retain the inner housing and be rotated in unison with the inner housing by the user when the outer housing and the inner housing are engaged;
(c) a compressible cleansing matrix attached to the outer housing and protruding into the inner housing, wherein the compressible cleansing matrix rotates with the outer housing; and
(d) a liquid disinfectant disposed in the compressible cleansing matrix.

2. A cleansing and capping device according to claim 1, that further comprises a removable lid to seal device from an external environment.

3. A cleansing and capping device according to claim 2, that has been sterilized.

4. A cleansing and capping device according to claim 1, wherein the inner housing includes at least one fluid path disposed on an inner surface of a sidewall.

5. A cleansing and capping device according to claim 1, wherein the compressible cleansing matrix comprises a liquid disinfectant, optionally isopropyl alcohol.

6. A cleansing and capping device according to claim 5, wherein the liquid disinfectant comprises a solution of about 70% isopropyl alcohol and water.

7. A cleansing and capping device according to claim 6, wherein the liquid disinfectant further comprises chlorhexidine, optionally chlorhexidine gluconate.

8. A cleansing and capping device according to claim 1, that further comprises a seal between the inner housing and the outer housing.

9. A cleansing and capping device according to claim 1, wherein the compressible cleansing matrix is configured to contact at least a valve surface of the threaded valve portion of the needle free connector when the device is threaded onto the threaded valve portion of the needle free connector.

10. A cleansing and capping device according to claim 9, wherein the compressible cleansing matrix is configured to also contact an additional surface of the threaded valve portion of the needlefree connector when the cleansing and capping device is threaded onto the threaded valve portion of the needlefree connector.

11. A cleansing and capping device according to claim 1, wherein the inner housing has a height that is less than the depth of the cavity of the outer housing.

12. A cleansing and capping device according to claim 1, wherein the outer housing further comprises a matrix well that extends into the cavity from the inner surface of the top of the outer housing to securely retain the compressible cleansing matrix, wherein an inner surface of the matrix well optionally includes one or more matrix retention splines disposed thereon.

13. An assembly that comprises a single piece of lidding material on to which a plurality of cleansing and capping devices according to claim 1, are sealed, optionally prior to sterilization of the assembly.

14. An assembly that comprises a cleansing and capping device according to claim 1, threaded on to a threaded valve portion of a needlefree connector.

15. A method of cleaning and/or disinfecting a needlefree connector, comprising:
(a) securing a cleansing and capping device according to claim 1, to a threaded valve portion of the needlefree connector; and
(b) after securing the cleansing and capping device to the needlefree connector, rotating the outer housing in relation to the inner housing, thereby cleansing one or more surfaces of the threaded valve portion of the needlefree connector.

16. A method according to claim 15, that further comprises removing the cleansing and capping device from the threaded valve portion of the needlefree connector within not less than about 30 seconds of attaching the cleansing and capping device to the needlefree connector.

17. A method according to claim 15, that further comprises leaving the cleansing and capping device connected to the threaded valve portion of the needlefree connector, optionally for up to 7 days.
